(19) United States
(12) Patent Application Publication
(10) Pub. No.: US 2024/0189616 A1
(43) Pub. Date: Jun. 13, 2024

(54) LIGHT IRRADIATION DEVICE AND MASSAGE APPARATUS INCLUDING SAME

(71) Applicant: BODYFRIEND CO., LTD., Seoul (KR)

(72) Inventors: Soo Hyun CHO, Seoul (KR); Jin Hwan KIM, Seoul (KR); Seung Hwa HONG, Seoul (KR); Dae Kwi PARK, Seoul (KR)

(21) Appl. No.: 18/386,856

(22) Filed: Nov. 3, 2023

(30) Foreign Application Priority Data
Dec. 7, 2022 (KR) 10-2022-0169257
Apr. 18, 2023 (KR) 10-2023-0050676

(51) Int. Cl. A61N 5/06; A61H 15/00; A61H 15/02

(52) U.S. Cl. CPC A61N 5/06; A61H 15/0078; A61H 15/02; A61H 2201/0149; A61H 2201/10; A61N 2005/0626; A61N 2005/0632

(57) ABSTRACT

A light irradiation device includes: a mount part having a slide rail; a slide part connected to the mount part and configured to slide along the slide rail; and an optical module provided on one side of the slide part to irradiate light onto a user's body. The mount part includes: a body part; a connection bracket detachably coupled to the body part; a lower mount housing coupled to the connection bracket; and an upper mount housing coupled to the slide rail, and having a slide slit formed to expose the slide rail to the outside.

### CROSS-REFERENCE TO RELATED APPLICATIONS

This application claims the priority of Korean Patent Application No. 10-2022-0169257 filed on Dec. 7, 2022, and No. 10-2023-0050676 filed on Apr. 18, 2023, in the Korean Intellectual Property Office, the entire contents of which is incorporated herein by reference.

### BACKGROUND OF THE INVENTION

### Field of the Invention

The present invention relates to a light irradiation device and a massage apparatus including the same, and more specifically, to a light irradiation device and a massage apparatus including the same, which can perform phototherapy on the scalp and the face while sliding, and a massage apparatus including the same.

### Background Art

Massage is a medical assistance therapy for controlling a user's physical modulation, helping blood circulation, and relieving fatigue of the user by applying various types of mechanical stimulation to a portion of the body, such as massaging, pressing, pulling, tapping, or moving a portion of the body of the user.

The growth in a massage demand caused an increase in demand for massage apparatuses, which provide an artificial massage function, because of economic feasibility and temporal reasons. That is, as the demand for relieving fatigue or stress is increased while loosening the agglomerated muscles through massage, various mechanical massage apparatuses for time and cost efficiencies are being released.

Recently, there is an increase in interest in health of people, and the massage apparatus is changing as an electronic device providing various additional functions, and/or medical functions from a device simply providing a massage function. Accordingly, researches on how to efficiently control a massage apparatus are continued.

### SUMMARY OF THE INVENTION

Accordingly, the present invention has been made to solve the above-mentioned problems occurring in the prior arts, and it is an object of the present invention to provide a light irradiation device and a massage apparatus including the same, which can perform phototherapy on the scalp and the face while sliding, and a massage apparatus including the same.

The objects of the present invention are not limited to the above-mentioned effects, and other effects, which are not specifically mentioned herein, will be clearly understood by those skilled in the art from the following description.

To accomplish the above object, according to the present invention, there is provided a light irradiation device including: a mount part having a slide rail; a slide part connected to the mount part and configured to slide along the slide rail; and an optical module provided on one side of the slide part to irradiate light onto a user's body.

In the light irradiation device according to an embodiment of the present invention, the mount part includes: a body part; a connection bracket detachably coupled to the body part; a lower mount housing coupled to the connection bracket; and an upper mount housing coupled to the slide rail, and having a slide slit formed to expose the slide rail to the outside.

In the light irradiation device according to an embodiment of the present invention, a connection bracket coupler into which the connection bracket is detachably inserted and coupled is protrudingly formed on the body part.

In the light irradiation device according to an embodiment of the present invention, a first retaining protrusion is formed on the outer circumferential surface of the connection bracket, and a second retaining protrusion, which selectively meshes with the first retaining protrusion 121 in a vertical direction based on a rotational angle of the connection bracket, is formed on the inner circumferential surface of the connection bracket coupler.

In the light irradiation device according to an embodiment of the present invention, multiple connection brackets with different heights are provided, and any one of the multiple connection brackets is detachably coupled to the body part corresponding to the user's height.

In the light irradiation device according to an embodiment of the present invention, the slide part includes a roller assembly which is connected to the slide rail and is provided to be slidable along the slide rail.

In the light irradiation device according to an embodiment of the present invention, a stopper is provided on the front end of the slide rail to prevent separation of the roller assembly.

In the light irradiation device according to an embodiment of the present invention, the slide part further comprises a position sensor arranged on at least one of the front end and the rear end of the slide rail to sense the position of the roller assembly.

In the light irradiation device according to an embodiment of the present invention, light intensity of light irradiated from the optical module is controlled based on position information of the roller assembly.

In the light irradiation device according to an embodiment of the present invention, the position sensor is any one among a microswitch, a reed switch, a proximity sensor, an optical sensor, and a hall sensor.

In the light irradiation device according to an embodiment of the present invention, an accommodation recess is formed at the front end of the mount part so that the optical module is accommodated in the accommodation recess when the slide part slides backward.

In another aspect of the present invention, there is provided a massage apparatus including: a body massage unit which massages at least a portion of a user's body; and a light irradiation device coupled to the body massage unit, wherein the light irradiation device irradiates light onto at least one of the scalp and the face of the user while sliding.

The light irradiation device and the massage apparatus including the same according to an embodiment of the present invention can selectively provide a scalp care or a face care while sliding.

### BRIEF DESCRIPTION OF THE DRAWINGS

FIG. 1 is a schematically perspective view of a massage apparatus according to an embodiment of the present invention.

FIG. 2 is a schematically perspective view of a light irradiation device according to an embodiment of the present invention.

FIG. 3 is a schematically bottom perspective view of the light irradiation device according to an embodiment of the present invention.

FIG. 4 is a schematically perspective view of a mount part.

FIG. 5 is a schematically bottom perspective view of the mount part.

FIG. 6 is a schematically exploded perspective view of the mount part.

FIG. 7 is a schematically exploded bottom perspective view of the mount part.

FIG. 8 shows (a) a view showing a coupling relation between a lower mount housing and a shielding member and (b) a sectional view take along the line A-A′ of FIG. 8(a).

FIG. 9 is a view showing a coupling relation between an upper mount housing and a slide rail of the mount part.

FIG. 10 shows (a) a view showing a coupling relation between the slide rail and a roller assembly and (b) a sectional view taken along the line B-B′ of FIG. 10(a).

FIG. 11 shows (a) a view showing a coupling relation between the slide rail and a position sensor and (b) a view showing a state in which the roller assembly is in contact with the position sensor.

FIG. 12 is a schematically bottom perspective view of the slide part.

FIG. 13 is a schematically side view of the slide part.

FIG. 14 is a schematically exploded perspective view of the slide part.

FIG. 15 is a schematically exploded bottom perspective view of the slide part.

FIG. 16 is a schematically perspective view of the roller assembly.

FIG. 17 shows (a) a view showing a state in which the roller assembly is coupled to the mount part and (b) a sectional view taken along the line C-C′ of FIG. 17(a).

FIG. 18 shows (a) a schematically perspective view of an optical module and FIG. (b) a schematically sectional view taken along the line D-D′ of FIG. 18(a).

FIG. 19 is a schematically sectional view of a light irradiation device according to an embodiment of the present invention.

FIG. 20 is a view showing a state in which the light irradiation device according to an embodiment of the present invention slides forward and backward.

### DETAILED DESCRIPTION OF THE PREFERRED EMBODIMENT

Advantages and features of the present invention and methods accomplishing the advantages and features will become apparent from the following detailed description of exemplary embodiments with reference to the accompanying drawings. However, the present invention is not limited to exemplary embodiment disclosed herein but will be implemented in various forms. The exemplary embodiments are provided so that the present invention is completely disclosed, and a person of ordinary skilled in the art can fully understand the scope of the present invention.

Terms used herein will be briefly described, and the disclosed embodiments will be described in detail.

The terms including descriptive or technical terms which are used herein should be construed as having meanings that are obvious to one of ordinary skill in the art. However, the terms may have different meanings according to an intention of one of ordinary skill in the art, precedent cases, or the appearance of new technologies. Also, some terms may be arbitrarily selected by the applicant, and in this case, the meaning of the selected terms will be described in detail in the detailed description of the invention. Thus, the terms used herein have to be defined based on the meaning of the terms together with the description throughout the specification.

The singular forms of the components may be understood into the plural forms unless otherwise specifically stated in the context. Furthermore, the plural forms may include singular forms unless otherwise specifically stated in the context.

In the entire specification of the present invention, when a certain part “includes” a certain component, other components are not excluded unless explicitly described otherwise, and other components may in fact be included.

In the present specification, an actuator refers to a configuration capable of providing a driving force. For example, the actuator may include a motor, a linear motor, an electronic motor, a DC motor, an AC motor, a linear actuator, an electric actuator, and the like, but is not limited thereto.

In this specification, a control unit may be implemented as a processor and a memory. The term “processor” should be interpreted broadly to encompass a general-purpose processor, a central processing unit (CPU), a microprocessor, a digital signal processor (DSP), a controller, a microcontroller, a state machine, and the like. In some environments, a “processor” may refer to an application specific integrated circuit (ASIC), a programmable logic device (PLD), a field programmable gate array (FPGA), or the like. The term “processor” may refer to a combination of processing devices, such as a combination of a DSP genome and a microprocessor, a combination of a plurality of microprocessors, a combination of one or more microprocessors in combination with a DSP couple core, or a combination of any other such configurations.

In the present specification, a massage apparatus may refer to a massage apparatus including a body massage unit and a leg massage unit. In addition, according to another embodiment, a body massage unit 2100 and a leg massage unit may be provided as separate devices (for example, a body massage apparatus and a leg massage apparatus), and the massage apparatus may refer to a body massage apparatus or a leg massage apparatus.

Hereinafter, embodiments of the present invention will be described in detail with reference to the accompanying drawings so as to be easily carried out by a person skilled in the art to which the present invention pertains. In addition, in order to clearly describe the present invention, portions irrelevant to the description will be omitted.

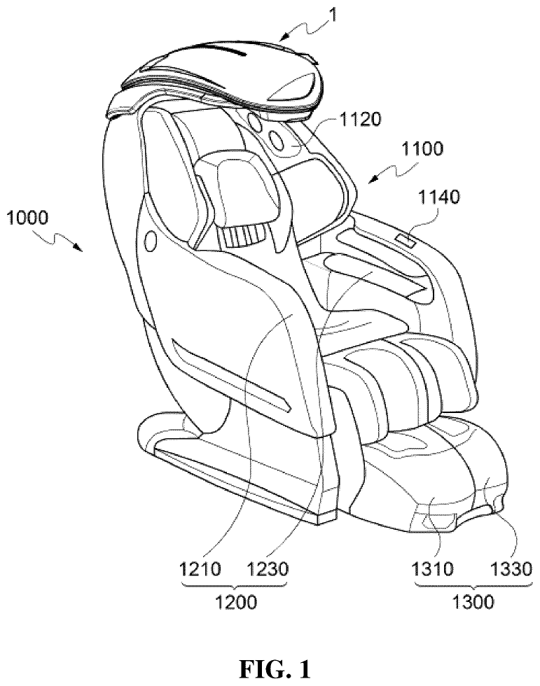

FIG. 1 is a schematically perspective view of a massage apparatus according to an embodiment of the present invention. Referring to FIG. 1, the massage apparatus 1000 according to an embodiment of the present invention forms an area for accommodating at least a portion of the body of a user, supports the user's body (for instance, the torso), and includes massage units 1100, 1200 and 1300 for massaging a portion of the body of the user, a light irradiation device 1 provided on one side of the massage units 1100, 1200 and 1300 to selectively provide a scalp care or a face care to the user, and a control unit which controls the overall operations of the light irradiation device 1.

The massage unit may include at least one of body massage units 1100 massaging at least a portion of the body of the user, an arm massage unit 1200 massaging the user's arms, and a leg massage unit 1300 massaging the user's legs.

The body massage unit 1100 can massage at least a portion of the body of the user. For instance, the body massage unit 1100 can massage at least a portion of either the user's upper body or lower body.

In detail, the body massage unit 1100 may include a massage module, which provides a massage function to at least a portion of the body of the user, an audio output module 1120, which provides any type of audio output to the user, a main frame which forms the framework of the body massage unit 1100 and supports the user's body, and a user input unit 1140 which receives any type of input from the user.

The components included in the body massage unit 1100 are just exemplary embodiments, and the body massage unit 1100 may include other various components.

Additionally, the shape and the structure of the massage apparatus 1000 illustrated in FIG. 1 are just examples, and various forms of massage apparatus 1000 can be included within the scope of the present invention without departing from the scope of the present invention defined by the claims of the present invention.

The body massage unit 1100 can form a space of any shape to accommodate the user. The body massage unit 1100 may have a space with the shape corresponding to the shape of the body of the user. For example, as illustrated in FIG. 1, the body massage unit 1100 can be realized as a seat type that can accommodate the user's whole body or a portion of the body and can support at least a portion of the body of the user. For example, the body massage unit 1100 may include a backrest supporting at least a portion of the user's upper body, and a seat supporting at least a portion of the user's lower body.

A portion of the body massage unit 1100 which contacts the ground may include any material or component (for example, a non-slip pad) for increasing frictional force, and may include wheels for enhancing mobility of the massage apparatus 1000.

At least a portion of the body massage unit 1100 can slide. For instance, when the body massage unit 1100 starts a massage (or when the body massage unit 1100 is activated), at least a portion of the body massage unit 1100 can slide forward. Additionally, the body massage unit 1100 can tilt backwards. As a result, the body massage unit 1100 can provide a massage while tilted backwards. For instance, the massage apparatus 1000 may include an actuator that tilts the massage apparatus 1000 or the body massage unit 1100 in the backward direction of the massage apparatus 1000 or the body massage unit 1100. The massage apparatus 1000 can activate the actuator to tilt the body massage unit 1100 or restore the body massage unit 1100 to the original state. In this instance, the actuator can be arranged in at least a portion of the massage apparatus (for example, the base frame).

According to an embodiment of the present invention, the massage apparatus 1000 may include at least one air cell (not illustrated). The air cell can be located in at least one area of the body massage unit 1100 (for instance, at least one of an area corresponding to the user's shoulder, an area corresponding to the pelvis, an area corresponding to the waist, out of the entire area of the body massage unit 1100), at least an area of the arm massage unit 1200, and at least an area of the leg massage unit 1300, but is not limited thereto, and may be arranged in various parts of the massage apparatus 1000.

The massage apparatus 1000 may include an air supply unit, which can inflate the air cell by supplying air to the air cell. The air supply unit may be located inside the body massage unit 1100 or may be located in the leg massage unit 1300. In addition, the air supply unit can be located outside the massage apparatus 1000.

The massage module 1110 may be installed inside the body massage unit 1100 to provide any type of mechanical stimulation to a user accommodated in the body massage unit 1100. As illustrated in FIG. 1, the massage module 1110 can move along the main frame 1130 installed inside the body massage unit 1100. The massage module 1110 can massage at least a portion of the body of the user while moving along the main frame 1130.

The main frame 1130 can form a frame of the internal configuration of the body massage unit 1100, and may be made of metal or plastic. For example, the main frame 1130 may be made of iron, alloy, steel, etc., but is not limited thereto, and can be realized with various hard materials.

The audio output module 1120 may be provided in various locations. For example, the audio output module 1120 may include multiple output units such as a top audio output unit arranged on the top of the seat part in contact with the user, a front audio output unit coupled to the front end of the arm massage unit 1200 on both sides of the seat part, and/or a rear audio output unit coupled to the rear end of the arm massage unit 1200, but is not limited thereto. In this case, the audio output module 1120 can provide stereophonic sound such as 5.1 channels, but is not limited thereto.

The arm massage unit 1200 can be arranged on one side or the other side of the body massage unit 1100. For example, the arm massage unit 1200 may include at least one among a first arm massage unit 1210 arranged on one side of the body massage unit 1100 to massage a user's one arm and a second arm massage unit 1230 arranged on the other side of the body massage unit 1100 to massage the user's other arm. The first arm massage unit 1210 can be arranged on at least one of the seat part and the backrest part of the body massage unit 1100. The second arm massage unit 1230 can be arranged on the other one of the seat part and the backrest part of the body massage unit 1100.

The leg massage unit 1300 can be arranged at the bottom of the body massage unit 1100 and/or at the front bottom of the massage apparatus 1000. The leg massage unit 1300 may include at least one among a first leg massage unit 1310 arranged on one side of the leg massage unit 1300 to massage the user's one leg and a second leg massage unit 1330 arranged on the other side of the leg massage unit 1300 to massage the user's other leg.

The leg massage unit 1300 includes a receiving space for accommodating the user's leg, and can provide a leg massage to the user by massaging at least a portion of the user's leg. For example, the leg massage unit 1300 may include a calf massage unit which massages the user's calf and/or a foot massage unit which massages the user's foot.

The leg massage unit 1300 can be adjustable in length according to the user's body characteristics. For example, when a user who is tall uses the massage apparatus 1000, it is necessary to extend the length of the leg massage unit 1300 since the user's calves are long. Moreover, when a user who is short uses the massage apparatus 1000, it is necessary to reduce the length of the leg massage unit 1300 since the user's calves are short. Accordingly, the leg massage unit 1300 can provide a leg massage tailored to the user's height. According to an embodiment of the present invention, the user can control the massage apparatus 1000 using a massage apparatus control device 2000. The massage apparatus control device 2000 can be connected to the massage apparatus 1000 through wired communication and/or wireless communication. The massage apparatus control device 2000 may include at least one user device (personal device), such as a remote controller, a cellular phone, and a PDA (Personal Digital Assistant), but is not limited thereto, and may include various electronic devices that can be connected to the massage apparatus 1000 through wired or wireless communication.

The control unit may be provided to control the overall operation of the massage apparatus. The control unit may include at least one processor. In a case in which the control unit includes a plurality of processors, at least a portion of the plurality of processors may be located at a physically spaced distance.

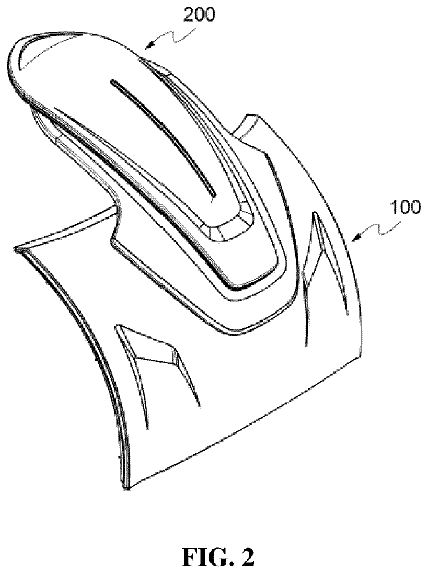

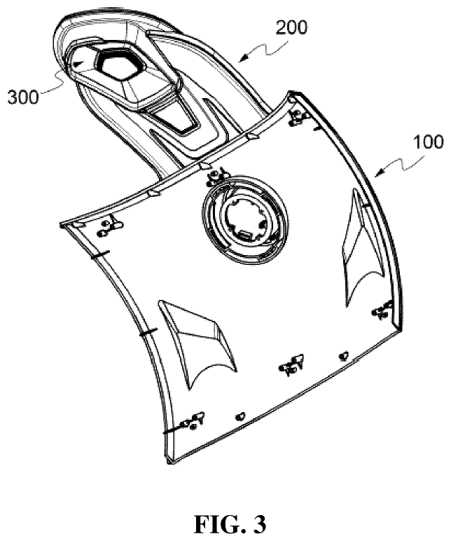

FIG. 2 is a schematically perspective view of a light irradiation device 1 according to an embodiment of the present invention, and FIG. 3 is a schematically bottom perspective view of the light irradiation device 1 according to an embodiment of the present invention. Referring to FIGS. 2 and 3, the light irradiation device 1 according to an embodiment of the present invention can be coupled to a massage apparatus 1000. For example, the light irradiation device 1 can be coupled to the upper rear of the body massage unit 1100.

The light irradiation device 1 can irradiate light onto the user's scalp or face while sliding to advance treatment. For instance, the light irradiation device 1 can provide the user with low level laser therapy (LLLT) that irradiates light of a specific wavelength onto the scalp or the face to promote metabolism in the corresponding area and activate tissue functions.

For example, the light irradiation device 1 may include a mount part 100, a sliding part 200, and an optical module 300.

The mount part 100 can be coupled to the body massage unit 1100 or can form a portion of the body massage unit 1100. In other words, the mount part 100 can be integrated with a rear cover of the body massage unit 1100.

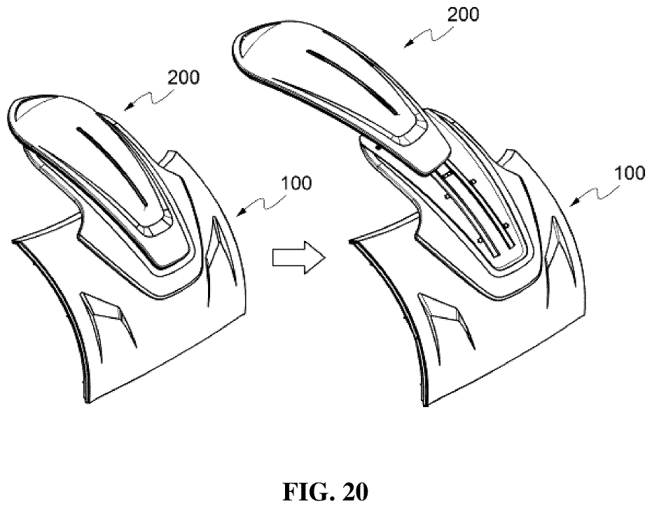

A slide part 200 may be connected to an upper portion of the mount part 100. The sliding part 200 can slide forward and backward in the state of being connected to the mount part 100 (see FIG. 20). When the sliding part 200 is positioned at the rear (scalp care mode, refer to the left drawing of FIG. 20), the light irradiation device 1 can irradiate light onto the user's scalp. When the sliding part 200 is positioned at the front (face care mode, refer to the right drawing of FIG. 20), the light irradiation device 1 can irradiate light onto the user's face. The user can grasp one side of the sliding part 200 and slide the slide part 200 forward or backward. Furthermore, an actuator can be provided in the sliding part 200 or the mount part 100 such that the sliding part 200 can be moved when the actuator is driven.

An optical module 300 is provided at one side of the slide part 200. For example, the optical module 300 can be equipped on the front end of the sliding part 200. The optical module 300 can include a light-emitting element, and can irradiate light to the user for treatment. Depending on the position of the sliding part 200, the optical module 300 can irradiate light of various wavelength ranges. For instance, if the sliding part 200 is located at the rear of the mount part 100 and the optical module 300 irradiates light onto the user's scalp, the optical module 300 can irradiate a light wavelength range effective for scalp care or preventing hair loss. Furthermore, if the sliding part 200 slides forward and is located at the front of the mount part 100, the optical module 300 can irradiate a light wavelength range effective for whitening or wrinkle care onto the user's face. Hereinafter, the detailed configuration of the light irradiation device 1 will be described with reference to the drawings.

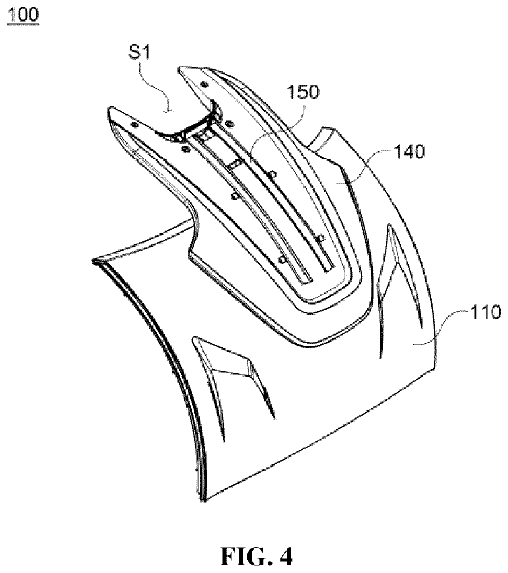

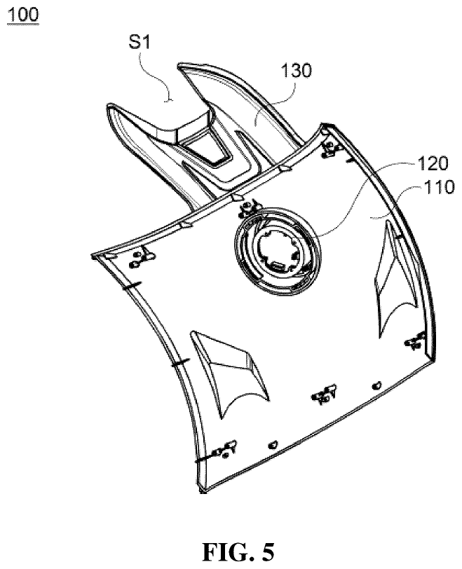

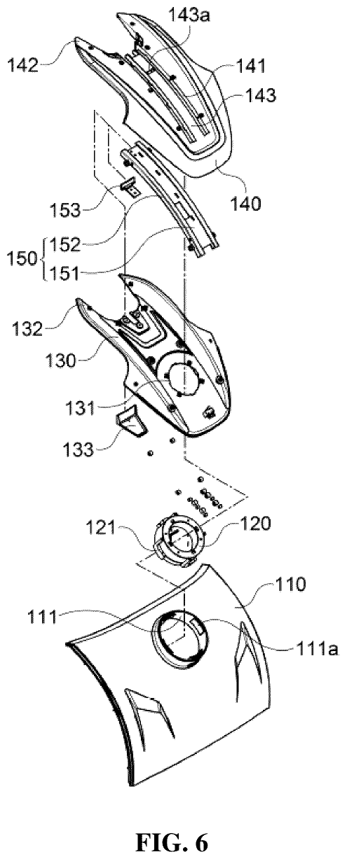

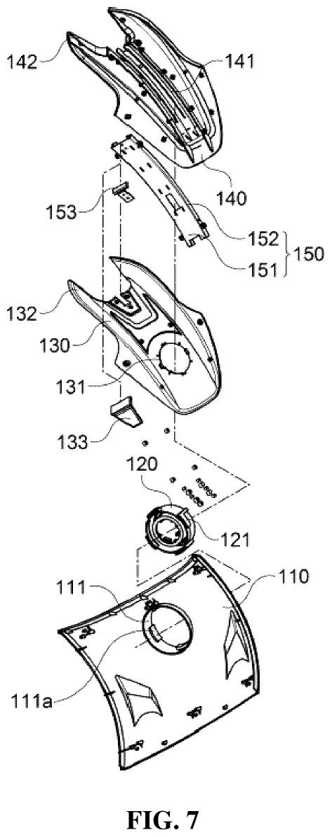

FIG. 4 is a schematically perspective view of a mount part, FIG. 5 is a schematically bottom perspective view of the mount part, FIG. 6 is a schematically exploded perspective view of the mount part, and FIG. 7 is a schematically exploded bottom perspective view of the mount part.

Referring to FIGS. 4 to 7, the mount part 100 can include a body part 110, a connection bracket 120, a lower mount housing 130, and an upper mount housing 140.

The body part 110 can be made of at least one of an injection molding material and a metallic material, and can be coupled to the body massage unit 1100. For instance, the body part 110 can be coupled to the upper rear of the body massage unit 110. Here, the body part 110 can be provided as one component of the body massage unit 1100. In other words, the body part 110 can form at least a portion of the rear cover of the body massage unit 1100.

The body part 110 may have a connection bracket coupler 111 having a hollow formed therein. The connection bracket coupler 111 can protrude upward or downward inside the body part 110. The connection bracket 120 can be detachably inserted and coupled to the connection bracket coupler 111. For this, a first retaining protrusion 121 can be protrudingly formed on the outer circumferential surface of the connection bracket 120, and a second retaining protrusion 111a, which selectively meshes in a vertical direction with the first retaining protrusion 121 based on a rotational angle of the connection bracket 120, can be protrudingly formed on the inner circumferential surface of the connection bracket coupler 111. In a state in which the connection bracket 120 is inserted into the connection bracket coupler 111 in a manner where the first retaining protrusion 121 of the connection bracket 120 and the second retaining protrusion 111a of the connection bracket coupler 111 do not overlap vertically, the connection bracket 120 is rotated such that the first retaining protrusion 121 and the second retaining protrusion 111a overlap vertically, so the connection bracket 120 can be fixed to the connection bracket coupler 111. Through a reverse process of the above-mentioned process, the connection bracket 120 can be separated from the connection bracket coupler 111. The structure for separably coupling the connection bracket 120 to the connection bracket coupler 111 can be substituted for structures commonly used in the technical field of the present invention, such as screw fastening, hook fastening, using a fastening member, etc.

The connection bracket 120 can connect the lower mount housing 130 and the body part 110. For instance, after the upper portion of the connection bracket 120 is coupled to the lower mount housing 130, the connection bracket 120 can be inserted and fixed into the connection bracket coupler 111 of the body part 110 to connect the lower mount housing 130 and the body part 110. A hollow may be formed inside the connection bracket 120. The hollow formed inside the connection bracket 120 can provide a space for arrangement of wires, flexible printed circuit boards, and other electronic elements.

The height of the connection bracket 120 can be adjusted based on the user's body condition. For instance, the connection bracket 120 may be multiple connection brackets 120 with different heights, and a connection bracket 120 corresponding to the user's height can be selected and mounted. If the user's sitting height is high, when the user sits on the massage apparatus 1000, there might be a problem that the head of the user touches the light irradiation device 1 or collides against the light irradiation device 1. In this case, a higher connection bracket 120 tailored to the user's height can be mounted on the light irradiation device 1. Conversely, if the user's sitting height is short, there might be a problem where the light projected from the light irradiation device 1 is not delivered to the body with sufficient intensity. In such a case, a shorter connection bracket 120 tailored to the user's height can be mounted on the light irradiation device. In other words, connection brackets with various heights can be manufactured/provided in various sizes according to the user's body condition, and the user can select and install the connection bracket of the appropriate height according to the user's height and use the light irradiation device. The lower mount housing 130 can be fastened to the connection bracket 120. For example, the lower mount housing 130 and the connecting bracket 120 can be fastened using separate fastening members or can be fixed through a mechanical structure. Additionally, the lower mount housing 130 and the connecting bracket 120 may be integrated as a single unit. For example, the lower mount housing 130 and the connecting bracket 120 can be integrally injection molded. An opening 131 can be formed in the lower mount housing 130. When the connecting bracket 120 is coupled to the lower mount housing 130, the opening 131 can be configured to communicate with the hollow inside of the connecting bracket 120. A first accommodation recess 132 which is depressed inwardly can be provided on the front end of the lower mount housing 130. The first accommodation recess 132 can provide a space where the optical module 300 is arranged.

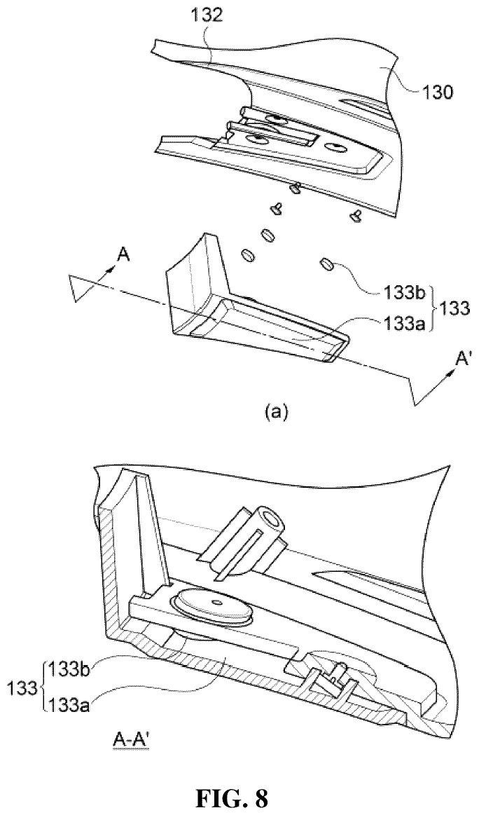

Referring to FIG. 8, a shielding member 133 can be detachably combined to the bottom surface of the lower mount housing 130. The shielding member 133 can be provided to prevent a tool, an electronic device, and the like arranged inside the lower mount housing 130 from being exposed to the outside. The shielding member 133 can include a shielding plate 133a and a magnet 133b. The shielding plate 133a can be configured as a structure that is overall bent in an ‘L’ shape. A magnet 133b can be provided on one side of the shielding plate 133a. The magnet 133b can be combined with a metal material, such as a screw, that is connected to the lower mount housing 130 through magnetic force. It is also possible to provide a metal material on one side of the shielding plate 133a and separately attach a magnet to the lower mount housing 130. Additionally, it is also possible to attach the shielding member 133 to the lower mount housing 130 using a separate fastening member or through a hook structure.

Referring to FIGS. 4 to 7, the upper mount housing 140 can be combined to an upper portion of the lower mount housing 130. In the upper mount housing 140, a slide slit 141 can be formed. The slide slit 141 can be formed by longitudinally cutting the inner side of the upper mount housing 140, and a slide rail 150 can be exposed to the outside through the slide slit. A roller assembly 230 of the slide part 200 can be connected to the slide rail 150 by passing through the slide slit 141. The slide slit 141 together with the slide rail 150 can provide a path for sliding of the slide part 200. That is, the slide part 200 can slide forward and backward along the slide slit 141.

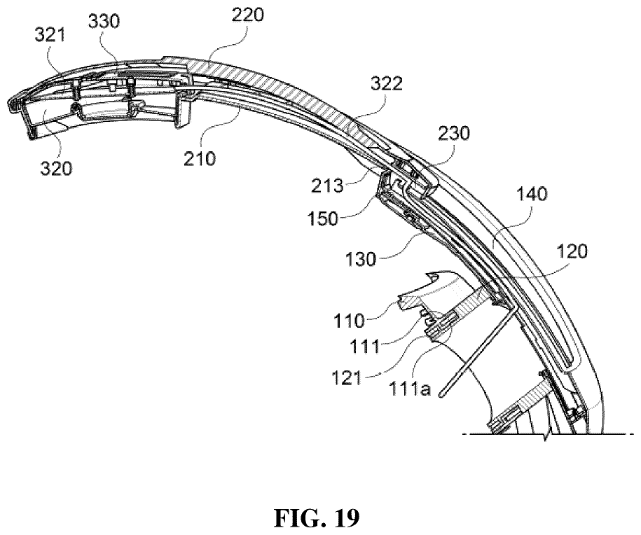

A flexible printed circuit board accommodation cover (or an accommodation space, or an accommodation part) 143 can be provided on the inner side of the slide slit 141. The flexible printed circuit board accommodation cover 143 can have a through hole 143a through which a flexible printed circuit board or a wire 322 (refer to FIG. 19) is introduced or withdrawn when the slide part 200 slides. The flexible printed circuit board accommodation cover 143 can provide a space where the flexible printed circuit board 322 is accommodated. Detailed description of the flexible printed circuit board accommodation cover 143 will be described later.

A second accommodation recess 140 which is depressed inwardly can be provided on the front end of the upper mount housing 140. The second accommodation recess 142 can be formed in a position corresponding to the first accommodation recess 132. The second accommodation recess 142 can provide a space where the optical module 300 is arranged. In other words, when the upper mount housing 140 is combined with the lower mount housing 130, due to the first accommodation recess 132 and the second accommodation recess 142, an accommodation recess S1 can be formed in the front end of the mount part 100. The accommodation recess S1 can provide a space where the optical module 300 is accommodated when the slide part 200 slides backward. That is, the optical module 300 can be accommodated inside the accommodation recess S1.

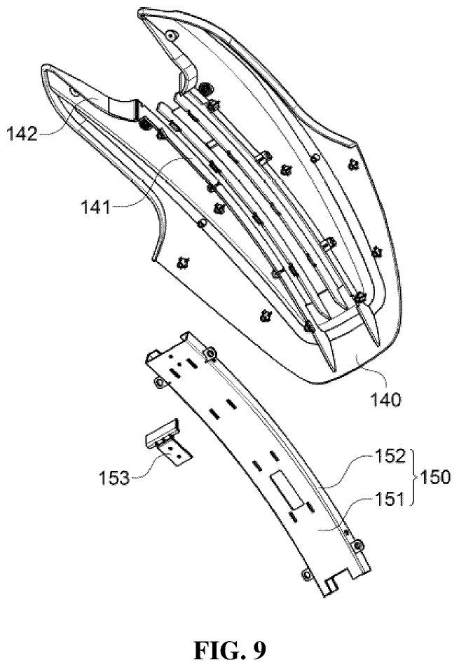

Referring to FIG. 9, the slide rail 150 can provide a movement path for the slide part 200, and can be coupled to the bottom surface of the upper mount housing 140. However, it is also possible to couple the slide rail 150 to the lower mount housing 130 as long as the slide rail provides a movement path for the slide part 200. The slide rail 150 can be made with a metal material or an injection molded part. The interior of the slide rail 150 can be exposed to the outside through the slide slit 141. Therefore, the roller assembly 230 can be connected to the slide rail 150 by passing through the slide slit 141.

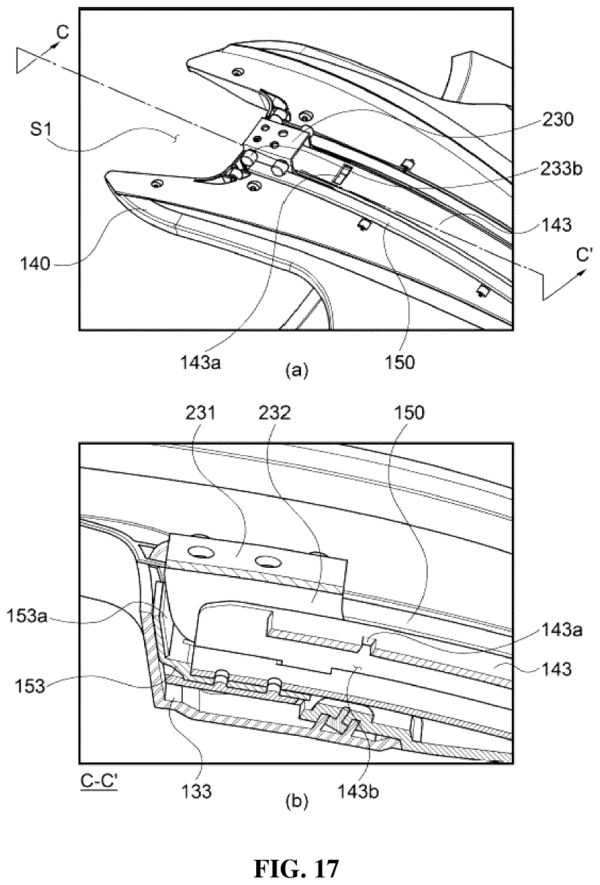

A stopper 153 can be provided on the front end of the slide rail 150 to prevent the roller assembly 230 from exiting the front of the slide rail 150. A cushioning member 153a (refer to FIG. 17) which can mitigate the impact when the roller assembly 230 collides can be provided on one side of the stopper 153 opposing to the slide rail 150.

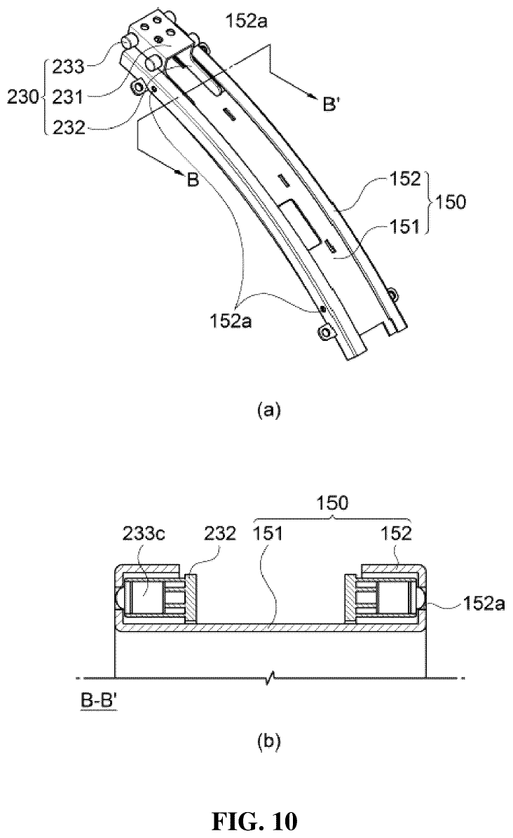

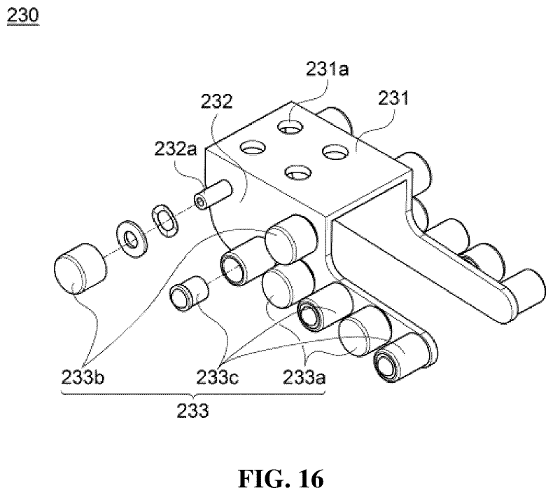

Referring to FIG. 10, the slide rail 150 can include a base part 151 extending forward and backward, and side parts 152 bent upwards from both sides of the base part 151 in the width direction. The side parts 152 can be bent several times. For example, the cross-sectional shape in the length direction of the side part 152 can overall have a ‘C’ shape. A first roller part 233a (refer to FIG. 16) of the roller assembly 230 can be arranged inside the side part 152, and a second roller part 233b (refer to FIG. 16) of the roller assembly 230 can be arranged on an upper portion of the side part 152.

The side part 152 can have at least one positioning hole 152a. For example, the positioning hole 152a may be provided in the front and rear of the side part 152. The positioning hole 152a can be provided to determine the position of the roller assembly 230. In other words, as the roller assembly 230 slides, when a roller assembly positioning member 233c of the roller assembly is inserted into the positioning hole 152a, the position of the roller assembly 230 can be determined. Thereafter, when a certain external force is applied to the roller assembly 230, the roller assembly positioning member 233c can be separated from the positioning hole 152a to be in a slidable state.

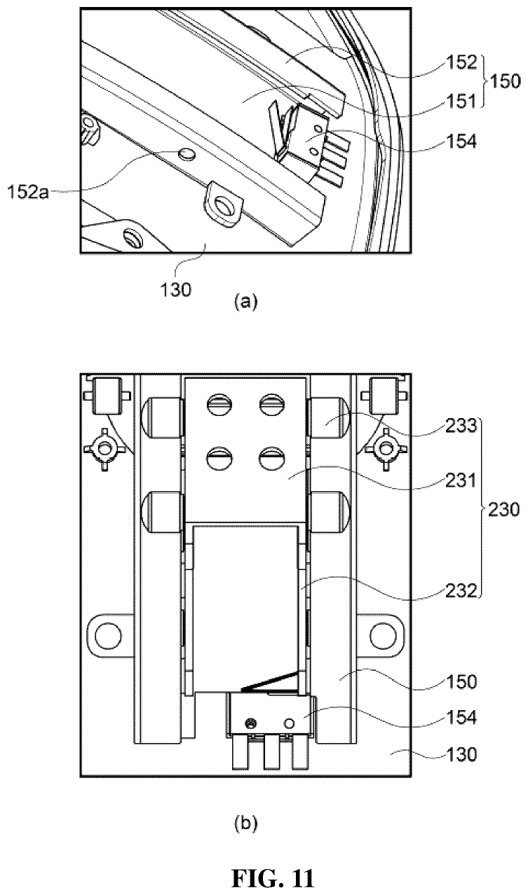

Referring to FIG. 11, a position sensor 154 can be arranged on at least one of the front end and the rear end of the slide rail 150 to sense the position of the roller assembly 230. For example, the position sensor 154 can be arranged at the rear end of the slide rail 150. The position sensor 154 can be directly coupled to the rear end of the slide rail 150 or can be arranged at the rear end of the slide rail 150 in a state of being coupled to the lower mount housing 130 or the upper mount housing 140.

The position sensor 154 may have, for example, at least one among a microswitch, a reed switch, a proximity sensor, an optical sensor, and a hall sensor. If the position sensor 154 is a microswitch, the position sensor 154 can detect whether the roller assembly 230 touches and determine whether the roller assembly 230 is positioned at the rear end or the front end of the slide rail.

If the position sensor 154 is a reed switch, the reed switch can be provided on the upper mount housing 140 corresponding to at least one of the front end and the rear end of the slide rail 150. For example, the reed switch can be provided on one side of the upper mount housing 140 corresponding to the front end of the slide rail 150 or on one side of the upper mount housing 140 corresponding to the rear end of the slide rail 150. However, it is also possible for the reed switch to be directly equipped on at least one of the front end and the rear end of the slide rail 150, or to be equipped on the lower mount housing 130 corresponding to at least one of the front end and the rear end of the slide rail 150. Meanwhile, a magnet capable of switching the reed switch on/off may be provided on the roller assembly 230. For example, the magnet can be fixed to the roller assembly 230 by a magnet bracket. When the roller assembly 230 slides along the slide rail 150 and the magnet provided on the roller assembly 230 is arranged to face the reed switch, the reed switch can be turned on. Moreover, when the magnet moves away from the reed switch, the reed switch can be turned off. Through the on/off status of the reed switch, the control unit can determine the position of the roller assembly 230.

The control unit can control the wavelength and light intensity of light irradiated from the optical module 300 based on position information of the roller assembly 230 received from the position sensor 154. For example, when the roller assembly 230 is located at the rear end of the slide rail 150 (in contact with the position sensor 154), the control unit can control the optical module 300 to irradiate light with a wavelength and intensity suitable for scalp care in a scalp care mode. Conversely, when the roller assembly 230 is located at the front end of the slide rail (not in contact with the position sensor 154), the control unit can control the optical module 300 to irradiate light with a wavelength and intensity suitable for facial care in a facial care mode. The light intensity (or strength of light) of light irradiated in the scalp care mode may be higher than that emitted in the facial care mode (for the purpose of user's visual protection).

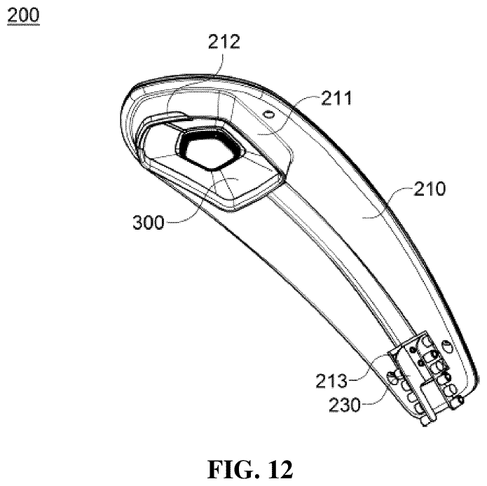

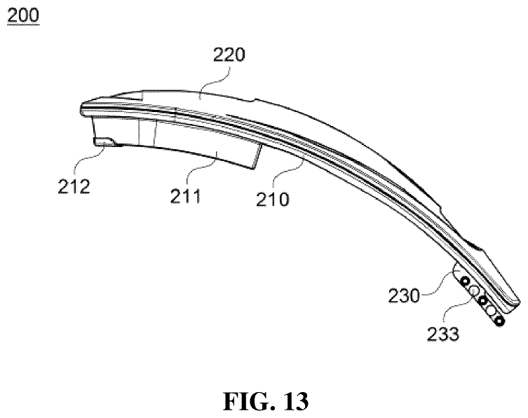

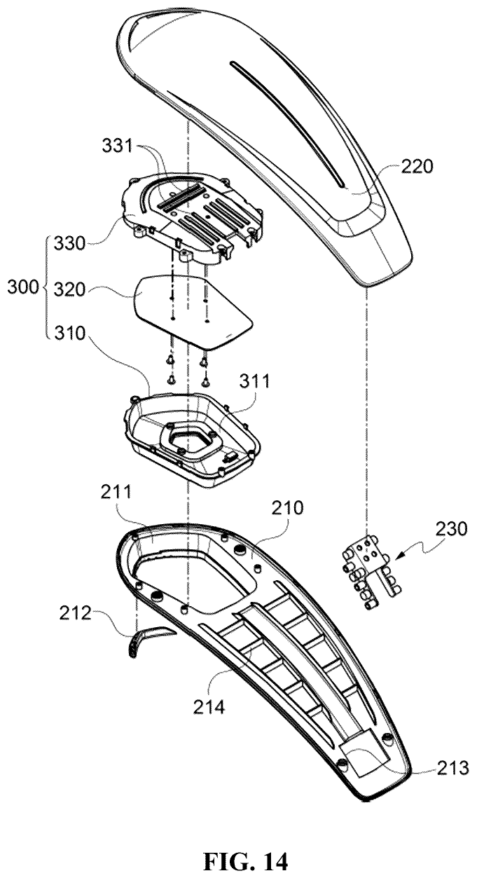

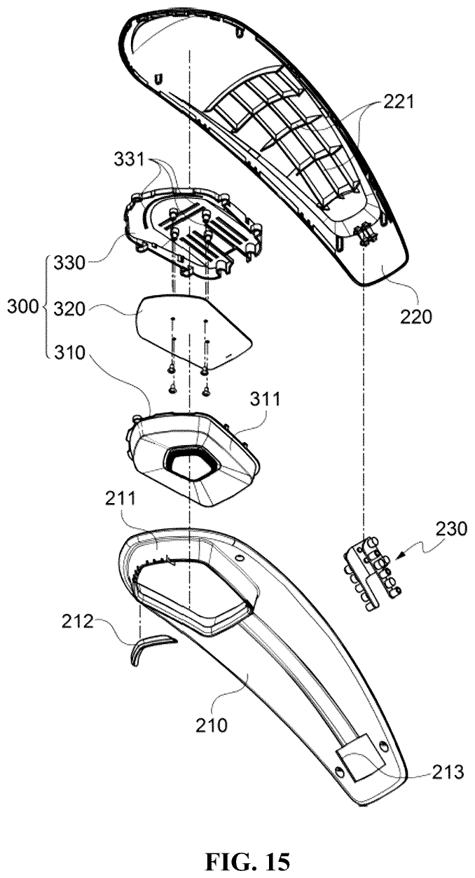

FIG. 12 is a schematically bottom perspective view of the slide part 200, FIG. 13 is a schematically side view of the slide part 200, FIG. 14 is a schematically exploded perspective view of the slide part, and FIG. 15 is a schematically exploded bottom perspective view of the slide part 200.

Referring to FIGS. 12 to 15, the slide part 200 may include a lower slide housing 210, an upper slide housing 220, and a roller assembly 230.

The lower slide housing 210 serves as a member constituting the lower portion of the slide part 200, and can be made with at least one of an injection-molded material and a metal material. An optical module coupler 211 may be provided on one side of the lower slide housing 210. For instance, the optical module coupler 211 can be provided on the front end of the lower slide housing 210. The optical module 300 can be inserted and coupled into the optical module coupler 211. Through the optical module coupler 211, the bottom surface of the optical module 300 can be exposed to the outside. The optical module coupler 211 can protrude a predetermined length below the lower slide housing 210. Therefore, the user can grasp the optical module coupler 211 and slide the slide part 200 forward or backward. For easy grasping by the user, a grip part 212 may be provided on the optical module coupler 211. For example, the grip part 212 can be provided on the lower front end of the optical module coupler 211. The grip part 212 can be made of a material that can prevent slipping. For instance, the grip part 212 can be made of a rubber material.

A roller assembly through hole 213 may be provided in the other side of the lower slide housing 210. The roller assembly 230 can penetrate through the roller assembly through hole 213 and be connected to the mount part 100. For example, the upper portion of the roller assembly 230 can be coupled to the upper slide housing 220, and the lower portion of the roller assembly 230 can penetrate through the roller assembly through hole 213 to be exposed to the outside. At least a portion of the externally exposed lower portion of the roller assembly 230 can be inserted and connected to the mount part 100. The lower slide housing 210 may include at least one first rigidity reinforcing rib 214. The first rigidity reinforcing rib 214 can be provided to enhance the mechanical rigidity of the lower slide housing 210 and can protrude from one side of the lower slide housing 210. Multiple first rigidity reinforcing ribs 214 can be provided to be spaced apart in the length or width direction of the lower slide housing 210. The arrangement of the first rigidity reinforcing rib 214 is not limited thereto, and can be provided at various positions of the lower slide housing 210 as long as it enhances the rigidity of the lower slide housing 210. In addition, the first rigidity reinforcing rib 214 can be integrated with the lower slide housing 210. In other words, the first rigidity reinforcing rib 214 can be injection-molded integrally with the lower slide housing 210. Moreover, the first rigidity reinforcing rib 214 can be separately manufactured using materials like resin, metal, etc., and then, be coupled to the lower slide housing 210.

The upper slide housing 220 is a member forming the upper portion of the slide part 200, and can have at least one of an injection molded material and a metal material. The upper slide housing 220 can be coupled to the upper surface of the lower slide housing 210. By coupling the upper slide housing 220 and the lower slide housing 210, the overall appearance of the slide part 200 can be formed. Inside the space formed between the upper slide housing 220 and the lower slide housing 210, an optical module 300, a flexible printed circuit board 322 (refer to FIG. 19), and an electronic element can be arranged. For example, the optical module 300 can be positioned at the front of the space formed between the upper slide housing 220 and the lower slide housing 210, and the flexible printed circuit board 322 can be configured to cross the inner space after being connected to the optical module 300 and to penetrate through the roller assembly through hole 213.

The upper slide housing 220 can include at least one second rigidity reinforcing rib 221. The second rigidity reinforcing rib 221 can be provided to enhance the mechanical rigidity of the upper slide housing 220 and can protrude from one side of the upper slide housing 220. Multiple second rigidity reinforcing ribs 221 can be arranged to be spaced apart in the length direction or width direction of the upper slide housing 220. Moreover, the second rigidity reinforcing rib 221 can be integrated with the upper slide housing 220. That is, the second rigidity reinforcing rib 221 can be injection molded together with the upper slide housing 220. Furthermore, the second rigidity reinforcing rib 221 can be separately manufactured using materials such as resin or metal, and then, combined with the upper slide housing 220.

The roller assembly 230 can be coupled to the upper slide housing 220. For instance, the roller assembly 230 can be coupled to the bottom surface of the upper slide housing 220. The roller assembly 230 can connect the upper slide housing 220 and the mount part 100. For example, one side of the roller assembly 230 can be coupled to the upper slide housing 220, and the other side can penetrate through the lower slide housing 210 and be connected to the slide rail 150 of the mount part 100. The roller assembly 230 can slide forward and backward along the slide rail 150. As the roller assembly 230 slides, the entire slide part 200 can slide on the mount part 100 (refer to FIG. 20).

Referring to FIG. 16, the roller assembly 230 can include, for example, a coupling panel 231, side panels 232, and rollers 233.

The coupling panel 231 can be coupled to the upper slide housing 220. For instance, at least one fastening hole 231a can be formed in the coupling panel 231, and a fastening member, such as a screw, can penetrate through the fastening hole 231a to be coupled to the upper slide housing 220. The method of coupling the coupling panel 231 to the upper slide housing 220 is not limited thereto, and can be changed in various ways commonly used.

The side panels 232 can be provided on both sides of the coupling panel 231 in the width direction. For example, the side panels 232 can be provided such that both sides of the coupling panel 231 in the width direction are bent downwards. Additionally, the side panels 232 can be separately manufactured and coupled to both sides of the coupling panel 231 in the width direction by a method such as welding. The lower end of each side panel 232 can be extended rearward in the lengthwise direction. Accordingly, the side panel 232 can be provided in an overall ‘L’ shape. By extending the lower end of the side panel 232 rearward in the lengthwise direction, the coupling force between the roller assembly 230 and the slide rail 150 can be enhanced.

At least one shaft 232a can be provided on the side panel 232. The shaft 232a can protrude outward in the width direction of the side panel 232. The roller 233 can be rotatably connected to the shaft 232a. To facilitate the rotation of the roller 233, at least one washer can be provided between the shaft 232a and the roller 233.

The roller 233 can be connected to the slide rail 150. For instance, the roller 233 may include at least one first roller part 233a provided on the lower portion of the side panel 232 and arranged on the inside of the slide rail 150, and a second roller part 233b provided on the upper portion of the side panel 232 and arranged on the upper side of the slide rail 150. Multiple first roller parts 233a and multiple second roller parts 233b can be provided at intervals in the lengthwise direction of the side panel 232.

The first roller part 233a can be arranged on the inside of the slide rail 150. For example, the first roller part 233a can be arranged on the inside of the side part 152 of the slide rail 150. The second roller part 233b can be arranged on the upper portion of the slide rail 150. For instance, the second roller part 233b can be arranged on the upper side of the side part 152 of the slide rail 150. By positioning the first roller part 233a and the second roller part 233b on the inside and the upper portion of the side part 152 of the slide rail 150 respectively, when the roller assembly 230 slides along the slide rail 150, the roller assembly 230 can be stably supported by the slide rails 150.

A roller assembly positioning member 233c, which is arranged on the inner surface of the slide rail 150, namely, on the inner surface of the side part, and presses against the inner surface of the slide rail 150, can be provided on the lower portion of the side panel 232. The roller assembly positioning member 233c can be, for example, a ball plunger. On the other hand, at least one positioning hole 152a can be formed in the side part 152 (see FIG. 10 for reference). For instance, the positioning hole 152a can be provided at the front end and the rear end of the side part 152. The positioning hole 152a can be provided to determine the position of the roller assembly 230. That is, as the roller assembly 230 slides, when the roller assembly positioning member 233c is inserted into the positioning hole 152a, the position of the roller assembly 230 can be determined. If a predetermined external force is applied to the roller assembly 230, the roller assembly positioning member 233c can be separated from the positioning hole 152a to be in a slidable state.

Referring to FIG. 17, a connection structure of the roller assembly 230 and the mount part 100 will be described. Referring to FIG. 17, the slide rail 150 can be exposed through the slide slit 141. At this time, a predetermined gap can be formed between the inner upper portion of the side part 152 of the slide rail 150 and the upper mount housing 140. The roller assembly 230 can be inserted into the slide rail 150 from the front of the upper mount housing 140 so that the side panel 232 is arranged in the gap. In this case, the roller assembly 230 can be inserted so that the first roller part 233a is positioned inside the side part 152 and the second roller part 233b is positioned on the upper portion of the side part 152.

When the roller assembly 230 is inserted into the slide rail 150, the stopper 153 can be coupled to the front end of the slide rail 150. The stopper 153 can prevent the roller assembly 230 from moving forward out of the slide rail 150. Furthermore, a cushioning member 153a which can mitigate an impact during collision with the roller assembly 230 is provided on one side of the stopper 153 facing the slide rail 150.

After the stopper 153 is coupled, the shielding member 133 can be detachably coupled to the bottom surface of the lower mount housing 130. The shielding member 133 can prevent tools like the stopper arranged inside the lower mount housing 130 and other electronic element from being exposed to the outside.

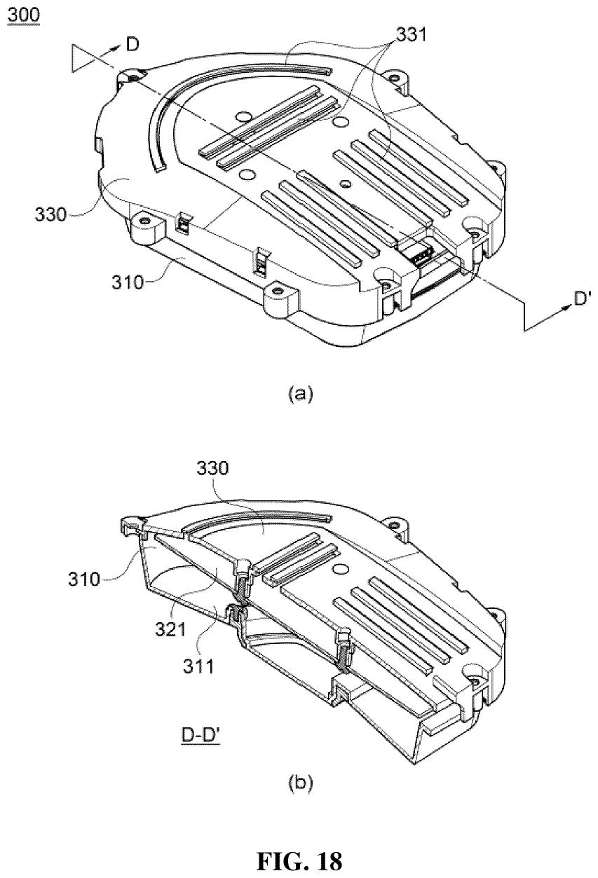

FIG. 18(a) is a schematically perspective view of the optical module, and FIG. 18(b) is a schematically sectional view taken along the line of D-D′ of FIG. 18(a).

Referring to FIG. 18, the optical module 300 can be provided to irradiate light onto the user's scalp or face. The optical module 300 can irradiate light of a specific wavelength to the user's scalp or face to stimulate metabolism in the corresponding area and activate tissue functions by exposing them to light of the specific wavelength.

The optical module 300 can include a diffusion plate 310, a light-emitting module 320, and a light-emitting module cover 330.

The diffusion plate 310 can be coupled to the lower slide housing 210. The bottom surface of the diffusion plate 310 can be exposed outside through the lower slide housing 210. In other words, when the diffusion plate 310 is coupled to the lower slide housing 210, the bottom surface of the diffusion plate 310 can be exposed outside through the optical module coupling part 211 of the lower slide housing 210. The diffusion plate 310 can evenly distribute the light produced by the light-emitting component and can eliminate hot spots. The bottom surface of the diffusion plate 310 may include an inclined surface 311 which is inclined upward from the edge of the diffusion plate 310 toward the center. Therefore, the diffusion plate 310 can be provided with a concave shape in the center.

The light-emitting module 320 can include at least one light-emitting element (not shown) and a printed circuit board 321. The light-emitting element can be provided, for example, as a light-emitting diode (LED). When the light-emitting element is provided as an LED, the light-emitting element can be combined with a red LED emitting red light and a blue LED emitting blue light.

Referring to FIG. 19, the printed circuit board 321 can be electrically connected to the control unit through the flexible printed circuit board 322. One end of the flexible printed circuit board 322 can be connected to the printed circuit board 321, and the flexible printed circuit board 322 can be arranged to pass through the inner space of the slide part 200, the roller assembly penetration part 213, and the mount part 100. For instance, one end of the flexible printed circuit board 322 can be connected to the printed circuit board 321 and the other end can pass through the inner space of the slide part 200 and the roller assembly penetration part 213 to enter the through hole 143a (refer to FIG. 17) of the flexible printed circuit board accommodation cover 143. The other end of the flexible printed circuit board 322 which has been inserted into the through hole 143a penetrates through the flexible printed circuit board accommodation space 143b (refer to FIG. 17) inside the flexible printed circuit board accommodation cover 143 and the connection bracket coupler 111, and finally, can be connected to the control unit. The flexible printed circuit board accommodation cover 143 can prevent the twisting of the flexible printed circuit board 322 when the slide part 200 slides along the slide rail 150. In other words, the flexible printed circuit board accommodation cover 143 can provide the flexible printed circuit board accommodation space 143b in which the flexible printed circuit board 322 can be inserted or withdrawn during the slide movement of the slide part 200, thereby preventing the twisting of the flexible printed circuit board 322. That is, when the slide part 200 moves to the rear end by sliding, the flexible printed circuit board 322 can be inserted into the flexible printed circuit board accommodation space 143b, and when the slide part 200 moves to the front end by sliding, the flexible printed circuit board 322 can be withdrawn from the flexible printed circuit board accommodation space 143b. Additionally, due to upward protruding anti-separation ribs provided on both sides of the flexible printed circuit board accommodation cover 143, the flexible printed circuit board can be stably inserted into and can get out of the flexible printed circuit board accommodation space 143b without departing from the accommodation cover.

The light-emitting module 320 can irradiate light of various wavelength bands corresponding to the position of the slide part 200. For instance, when the slide part 200 is located at the rear of the mount part 100 and irradiates light to the user's scalp (scalp care mode), the light-emitting module 320 can irradiate the user's scalp with light in a wavelength band effective for scalp care or hair loss prevention. Furthermore, when the slide part 200 slides forward and is positioned at the front of the mount part 100 (facial care mode), the light-emitting module 320 can irradiate light onto the user's face in a wavelength band effective for whitening or wrinkle care. For example, the light-emitting module 320 can selectively irradiate light with the wavelength bands of 420 to 480 nm, 630 to 690 nm, or 850 to 940 nm onto the user's scalp or face.

The light-emitting module cover 330 can be coupled to the diffusion plate 310. The light-emitting module cover 330 can prevent the light-emitting module 320 from being contaminated by foreign substances or damaged by an external impact. At least one heat dissipation slit (331) can be formed in the light-emitting module cover 330.

Although the present invention has been described with reference to exemplary embodiments along with the accompanying drawings, it should be understood that this is merely exemplary and not intended to limit the present invention. Furthermore, it is evident to those skilled in the art that various modifications and variations can be made within the scope of the technical concept of the present invention without departing from the scope thereof.

Additionally, the configuration and functions of the massage device in this specification can be expanded and applied to various (wearable) healthcare devices capable of providing healthcare functions (e.g., massage functions, biometric signal measurement functions, etc.). Therefore, the term ‘massage device’ in this specification can be rearranged with ‘(wearable) healthcare device’ as well.

What is claimed is:

1. A light irradiation device comprises:
a mount part having a slide rail;
a slide part connected to the mount part and configured to slide along the slide rail; and
an optical module provided on one side of the slide part to irradiate light onto a user's body.

2. The light irradiation device according to claim 1, wherein the mount part comprises:
a body part;
a connection bracket detachably coupled to the body part;
a lower mount housing coupled to the connection bracket; and
an upper mount housing coupled to the slide rail, and having a slide slit formed to expose the slide rail to the outside.

3. The light irradiation device according to claim 2, wherein a connection bracket coupler into which the connection bracket is detachably inserted and coupled is protrudingly formed on the body part.

4. The light irradiation device according to claim 3, wherein a first retaining protrusion is formed on the outer circumferential surface of the connection bracket, and a second retaining protrusion, which selectively meshes with the first retaining protrusion 121 in a vertical direction based on a rotational angle of the connection bracket, is formed on the inner circumferential surface of the connection bracket coupler.

5. The light irradiation device according to claim 2, wherein multiple connection brackets with different heights are provided, and
wherein any one of the multiple connection brackets is detachably coupled to the body part corresponding to the user's height.

6. The light irradiation device according to claim 1, wherein the slide part comprises a roller assembly which is connected to the slide rail and is provided to be slidable along the slide rail.

7. The light irradiation device according to claim 6, wherein a stopper is provided on the front end of the slide rail to prevent separation of the roller assembly.

8. The light irradiation device according to claim 6, wherein the slide part further comprises a position sensor arranged on at least one of the front end and the rear end of the slide rail to sense the position of the roller assembly.

9. The light irradiation device according to claim 8, wherein light intensity of light irradiated from the optical module is controlled based on position information of the roller assembly.

10. The light irradiation device according to claim 8, wherein the position sensor is any one among a microswitch, a reed switch, a proximity sensor, an optical sensor, and a hall sensor.

11. The light irradiation device according to claim 1, wherein an accommodation recess is formed at the front end of the mount part so that the optical module is accommodated in the accommodation recess when the slide part slides backward.

12. A massage apparatus comprising:
a body massage unit which massages at least a portion of a user's body; and
a light irradiation device according claim 1, which is coupled to the body massage unit,
wherein the light irradiation device irradiates light onto at least one of the scalp and the face of the user while sliding.
